(19) United States
(12) Patent Application Publication
(10) Pub. No.: US 2024/0293352 A1
(43) Pub. Date: Sep. 5, 2024

(54) PHARMACEUTICAL COMPOSITIONS COMPRISING VANADIUM SALTS

(71) Applicants: Ottawa Hospital Research Institute, Ottawa (CA); Colorado State University Research Foundation, Fort Collins, CO (US)

(72) Inventors: Jean-Simon Diallo, Ottawa (CA); Debbie C. Crans, Berthoud, CO (US); Rozanne Arulanandam, Orleans (CA)

(73) Assignees: Ottawa Hospital Research Institute, Ottawa, ON (CA); Colorado State University Research Foundation, Fort Collins, CO (US)

(21) Appl. No.: 18/288,617

(22) PCT Filed: May 6, 2022
(86) PCT No.: PCT/CA2022/050713

§ 371 Date: Oct. 27, 2023

Related U.S. Application Data
(60) Provisional application No. 63/185,594, filed on May 7, 2021.

(51) Int. Cl. A61K 31/28; A61K 33/42; A61K 35/766; A61K 35/768; A61P 35/00

(52) U.S. Cl. CPC A61K 31/28; A61K 33/42; A61K 35/766; A61K 35/768; A61P 35/00

(57) ABSTRACT

The present application relates to vanadium compounds. More specifically, the present application relates to pharmaceutical compositions comprising vanadium salts, use thereof and methods using the same. The present application also includes compositions comprising vanadium compounds and a therapeutic or prophylactic virus providing for increased virus infection, spread, titer, activity, cytotoxicity and/or immunotherapeutic activity.

The present application claims priority to U.S. Provisional Patent Application No. 63/185,594, which was filed May 7, 2021, the contents of which are incorporated herein by reference in their entirety.

### FIELD

The present application is in the field of vanadium compounds. More specifically, the present application relates to pharmaceutical compositions comprising vanadium salts.

### BACKGROUND

Genetically attenuated viruses form the basis of a growing number of biotechnology and pharmaceutical platforms. Emerging in the field of cancer therapeutics, oncolytic virotherapy has shown significant promise over the last decade. A number of oncolytic viruses (OV) based on a wide range of viral backbones from small RNA viruses (e.g. rhabdoviruses), to large DNA viruses (e.g. poxviruses, herpesviruses) are currently being evaluated in clinical trials to treat a range of cancer types. Generating substantial excitement for this form of cancer therapy, approval of the first-in-class OV based on herpes-simplex virus-1 (HSV-1) for treatment of melanoma was granted by the FDA in 2015.

Oncolytic viruses (OVs) are, typically, self-amplifying biotherapeutic agents that have been selected or engineered to preferentially infect and kill cancer cells. When effective, OVs lead to tumor eradication not only by direct lysis of cancer cells but also through downstream generation of anti-cancer immune responses, vascular shutdown, and therapeutic transgene expression. As a basis for their selectivity, OVs exploit cellular defects that are inherent to the cancerous phenotype. This includes dysfunctional anti-viral responses and immune evasion, increased cell proliferation and metabolism, and leaky tumor vasculature. The biological environment ensuing from tumorigenesis is well suited to support the growth of genetically attenuated OVs that are otherwise harmless to normal cells.

OVs stand to be an attractive therapeutic modality for cancer because of their curative potential and their relatively mild side effects amounting to acute flu-like symptoms. However, the capacity of an oncolytic virus to both replicate and stimulate an anti-tumor immune response is a recognized hurdle to achieving the desired anti-cancer effects.

Many approaches exist to enhance the activity of oncolytic viruses. One of the most common is the use of therapeutic transgenes encoded within and expressed by the virus. The limitation with this approach is that it relies heavily on the capacity of the virus to replicate and spread in the tumor in order to express the transgene, which can be minimal in some tumors. Immune checkpoint inhibitors are recognized to enhance oncolytic virus activity by overcoming the “brakes” on anti-tumor immunity. These do not affect the spread or oncolytic activity of the virus itself, including its inherent capacity to induce anti-tumor immune responses, particularly innate immune responses. Many small molecules have been described that improve the efficacy of oncolytic viruses. Each has their specific range of applications and effects. Most, including well-known drugs like cyclophosphamide, are highly immunosuppressive. Very few are known to be immunostimulatory. These include anthracyclines like doxorubicin and mitoxantrone for example, or SMAC mimetics LCL-161 and Birinapant. However, these drugs do not have the capacity to enhance viral spread.

It has recently been demonstrated that vanadyl sulfate, orthovanadate, and bismaltolatoxovanadate (BMOV) improve the replication and spread of oncolytic RNA viruses including vesicular stomatitis virus (VSV) and measles while simultaneously enhancing immune stimulation (Selman et al, Mol Ther, 2018 and patent application PCT/CA2017/051176). Vanadium compounds have been historically studied for their anti-diabetic effects through hyperphosphorylation of the insulin receptor. While safe, derivatives such as bis-ethylato-oxovanadate (BEOV) have been evaluated and failed in human phase II clinical trials as antidiabetics. Vanadyl sulfate also has been tested clinically for the treatment of diabetes, but has yet to be approved as an anti-diabetic (phase III clinical trial registered completed NCT00561132).

Currently, oncolytic viruses (OVs) are mainly delivered intratumorally in patients. The first and as of yet only approved OV product in North America and Europe, Imlygic™ is administered in multiple superficial cancerous lesions in the context of advanced melanoma. Intravenous delivery of OVs still remains a hurdle as substantial amounts of virus are captured by sink organs like the liver and lungs, or neutralized by blood and immune cells, or hindered by poor vascularization and other tumor microenvironment associated barriers. Until such hurdles are overcome to allow for effective systemic delivery of OVs, there remains a significant need for methods and novel compositions for improving the effectiveness of intratumoral delivery of OVs, in such a way that would not be additionally onerous to clinicians that are administering these drugs.

Given their capacity to enhance viral spread and promote anti-tumor immunity simultaneously, the co-administration of vanadium-based molecules and OVs as a single injectable product would therefore be an attractive approach to enhance the spread and efficacy of OVs locally within the tumor. Co-administration is desirable over separate administration of the vanadium compound and the OV because separate administration requires clinicians to administer the vanadium-based molecules by injecting all of the solid tumors of the patient, and then do the same with the OV, suitably in the same exact location. This is both cumbersome for the clinician and uncomfortable for the patient. Accordingly, it would be desirable to co-formulate a composition with both the OV and the vanadium-based molecule so that the clinician only needs to inject once per lesion and the tumor receives the OV and vanadium-based molecule in the same area.

While desirable from a practical and clinical standpoint, there are multiple challenges to co-formulating a composition with both the OV and the vanadium-based molecule. First, co-formulation requires that the compound and virus are biochemically compatible in order to permit admixing of virus and compound prior to intratumoral injection of the mixture. In the most extreme, the compound and virus would be manufactured as a combination product requiring stability over several months to years. Alternately, compound and virus can be produced and stored separately for admixing prior to injection, which in a clinical setting would nevertheless require stability for potentially several hours prior to injection. A second limitation, not mutually exclusive to the first, is dosing. Owing to the requirement for high concentrations of the vanadium compound in an ad-mixed virus co-formulation, there is a need for the vanadium compound to be more potent and not precipitate at the necessary high concentrations of both virus and compound.

As such, there is need to provide improved compounds and compositions that enhance oncolytic virus growth, spread, and/or cytotoxicity. Compounds and compositions that enhance virotherapy-induced anti-tumor immune responses and/or that increase anti-cancer efficacy are also desired in the field. Improved or alternative methods for treating cancer cells in vitro and in vivo are also desired.

### SUMMARY

It has been surprisingly shown herein that compounds and compositions of the present application comprising vanadium compounds provide for enhanced virus infection, spread, titer, activity, cytotoxicity and/or immunotherapeutic activity. Comparable compounds and compositions did not display the same properties, highlighting the surprising results obtained with the compounds and compositions of the application.

Accordingly, the present application includes a pharmaceutical composition comprising one or more vanadium compounds wherein the one or more vanadium compounds are selected from a citrate salt of vanadium, a phosphate salt of vanadium or mixed salts thereof.

Further provided is a use of one or more vanadium compounds of the present application for increasing the infection, spread, titer, activity, cytotoxicity and/or immunotherapeutic activity of a virus.

Also included is a use of one or more vanadium compounds present application in the manufacture of a medicament for the treatment of a cancer in a subject in need thereof.

Use of one or more vanadium compounds present application as an adjuvant for virus-based vaccine is further included.

The present application also includes a method of treating a cancer or tumor, comprising administering an effective amount a pharmaceutical composition of the present application to a cell or subject in need thereof.

The present application further provides a method of increasing infection, spread, titer, activity, cytotoxicity and/or immunotherapeutic activity of one or more viruses, comprising administering, to cells or a subject in need thereof, an effective amount of one or more vanadium compounds of the application in combination with an effective amount of the one or more viruses.

The present application further includes a method for increasing permissiveness of cells to one or more viruses, comprising administering to the cells an effective amount of one or more vanadium compounds of the application in combination with an effective amount of the one or more viruses.

Further provided is a kit comprising: one or more vanadium compounds of the present application; and one or more viruses.

Other features and advantages of the present application will become apparent from the following detailed description. It should be understood, however, that the detailed description and the specific examples, while indicating embodiments of the application, are given by way of illustration only and the scope of the claims should not be limited by these embodiments, but should be given the broadest interpretation consistent with the description as a whole.

### BRIEF DESCRIPTION OF DRAWINGS

The embodiments of the application will now be described in greater detail with reference to the attached drawings in which:

FIG. 1 is a graph of the viral titers of VSV strain D51 (VSVD51) measured after 60 minutes incubation in the presence of vanadate and vanadyl sulfate, and exemplary vanadium sulfate citrate solution (CDOS136), vanadium sulfate phosphate solution (CDOS137), or vanadium-citrate compounds (CDOS139, CDOS140) or in PBS (VSV) as a control, at room temperature. VSVD51 titers were determined by the number of plaque-forming units per ml (pfu/ml) of solution.

FIG. 2 shows the appearance and pH of solutions of vanadyl sulfate resuspended at a concentration of 16 mg/ml in a) phosphate buffered saline (PBS); b) 2M HEPES (4-(2-hydroxyethyl)piperazine-1-ethanesulfonic acid, N-(2-hydroxyethyl)piperazine-N′-(2-ethanesulfonic acid)) buffered at pH 7.4; c) 2M Tris (tris(hydroxymethyl)aminomethane) buffered at pH 7.4; and d) PBS, with pH neutralized with NaOH.

FIG. 3A and FIG. 3B show the chemical structure of vanadate and vanadyl sulfate, respectively, according to exemplary embodiments of the present application. FIG. 3C and FIG. 3D show the impact of varying concentrations of exemplary vanadate and vanadyl sulfate, respectively on VSVD51-GFP oncolytic activity in 786-0 cells after 48 h according to exemplary embodiments of the application. VSVD51-GFP is provided at a multiplicity of infection of 0.01 after 4 h treatment with the compounds. Relative metabolic activity (compared to untreated 786-0 cells) is measured using resazurin is indicated on the left y-axis, whereas GFP foci are measured using fluorescence image analysis following high-throughput microscopy using a Cellomics ArrayScan™ (right y-axis). Hashed line indicates approximate concentration at which peak effect on GFP foci has been attained.

FIG. 4A and FIG. 4B show the impact of varying concentrations of a solution of vanadium(IV) sulfate buffered to pH 7.4 with exemplary sodium citrate (CDOS136); or exemplary sodium phosphate (CDOS137), respectively, on VSVD51-GFP oncolytic activity in 786-0 cells after 48 h. VSVD51-GFP is provided at a multiplicity of infection of 0.01 after 4 h treatment with the compounds. Relative metabolic activity (compared to untreated 786-0 cells) is measured using resazurin is indicated on the left y-axis, whereas GFP foci are measured using fluorescence image analysis following high-throughput microscopy using a Cellomics ArrayScan™ (right y-axis). Hashed line indicates approximate concentration at which peak effect on GFP foci has been attained.

FIG. 5A, FIG. 5B and FIG. 5C show the chemical structure of CDOS139-V(IV) citrate; CDOS140-V(V) citrate; and CDOS141-V(V) citrate peroxo, respectively, according to exemplary embodiments of the present application. FIG. 5D, FIG. 5E and FIG. 5F show the impact of varying concentrations of CDOS139; CDOS140; and CDOS141, respectively on VSVD51-GFP oncolytic activity in 786-0 cells after 48 h, according to exemplary embodiments of the application. VSVD51-GFP is provided at a multiplicity of infection of 0.01 after 4 h treatment with the compounds. Relative metabolic activity (compared to untreated 786-0 cells) is measured using resazurin is indicated on the left y-axis, whereas GFP foci are measured using fluorescence image analysis following high-throughput microscopy using a Cellomics ArrayScan™ (right y-axis). Hashed line indicates approximate concentration at which peak effect on GFP foci has been attained.

FIG. 6 shows vanadium IV/V-citrate preparations (CDOS148, CDOS150, and CDOS155) and their resolved structures according to exemplary embodiments of the present application.

FIG. 7 shows exemplary vanadium IV/V-citrate preparations (CDOS148, CDOS150, and CDOS155) at neutral pH tested for their ability to enhance the growth of VSVd51-GFP in CT26 wt colon carcinoma cells as measured by GFP expression, indicating viral growth and spread, according to exemplary embodiments of the present application.

FIG. 8 shows the normalized weights of mice over time at the different dosing levels of exemplary vanadium IV/V-citrate preparations (CDOS148, CDOS150, and CDOS155), according to exemplary embodiments of the present application.

FIG. 9 shows test results of the ability of exemplary V-(IV)-citrate compound CDOS155 to be co-formulated, using two different derivatives of VSVd51: encoding Firefly luciferase (VSV-FLuc) and encoding the IL12 pro-inflammatory cytokine (VSV-IL12), according to exemplary embodiments of the present application.

FIG. 10 shows exemplary CDOS155 used at 10 mg/kg when administered intratumorally and separately (15 minutes apart) from VSV-Fluc (1E8 plaque forming units, PFU) enhanced the antitumor effects of VSV-Fluc to a comparable extent to a similar regimen of vanadyl sulfate (50 mg/kg), according to exemplary embodiments of the present application.

FIG. 11 shows the potential of exemplary CDOS155 to be co-formulated with VSV-IL12, using Ct26 wt syngeneic tumor model, where a co-formulation of CDOS155 was prepared with VSV-IL12 resulting in a single injectable co-formulated dose of 10 mg/kg CDOS155: 1E8 PFU VSV-IL12 and the co-formulation administered three times (days 0, 2, 4), according to exemplary embodiments of the present application.

### DETAILED DESCRIPTION

### I. Definitions

Unless otherwise indicated, the definitions and embodiments described in this and other sections are intended to be applicable to all embodiments and aspects of the present application herein described for which they are suitable as would be understood by a person skilled in the art.

As used in this application and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

The term “consisting” and its derivatives as used herein are intended to be closed terms that specify the presence of the stated features, elements, components, groups, integers, and/or steps, and also exclude the presence of other unstated features, elements, components, groups, integers and/or steps.

The term “consisting essentially of”, as used herein, is intended to specify the presence of the stated features, elements, components, groups, integers, and/or steps as well as those that do not materially affect the basic and novel characteristic(s) of these features, elements, components, groups, integers, and/or steps.

The terms “about”, “substantially” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies or unless the context suggests otherwise to a person skilled in the art.

As used in the present application, the singular forms “a”, “an” and “the” include plural references unless the content clearly dictates otherwise. For example, an embodiment including “a compound” should be understood to present certain aspects with one compound, or two or more additional compounds.

In embodiments comprising an “additional” or “second” component, such as an additional or second compound, the second component as used herein is chemically different from the other components or first component. A “third” component is different from the other, first, and second components, and further enumerated or “additional” components are similarly different.

The term “and/or” as used herein means that the listed items are present, or used, individually or in combination. In effect, this term means that “one or more of” or “one or more” of the listed items is used or present. The term “and/or” with respect to enantiomers, prodrugs, salts and/or solvates thereof means that the compounds of the application exist as individual enantiomers, prodrugs, salts and hydrates, as well as a combination of, for example, a salt of a solvate of a compound of the application.

The term “vanadium compound of the application” or “vanadium compound of the present application” and the like as used herein refers to a phosphate or citrate salt of vanadium, or a combination phosphate and citrate salt of vanadium.

The term “vanadium compound” as used herein refers to compounds which include a vanadium transition metal core. The vanadium core can be in any oxidation state.

The term “citrate” as used herein refers to salts of citric acid. Citric acid has the following structure:

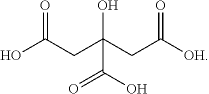 [SMILES: O=C(O)CC(O)(CC(=O)O)C(=O)O]

Citrate may present different protonation states depending on pH of the solution or methods of its preparation, and salts can be formed by replacing the acidic protons with one, two, three or four cations. A person skilled in the art would understand that vanadium can form a coordination bond with one, two, three or all four of the oxygen atoms in the OH groups of citric acid.

The term “phosphate” as used herein refers to salts of a phosphoric acid, most commonly the salts of orthophosphoric acid. Orthophosphoric acid has the following structure:

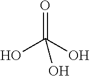

and salts can be formed by replacing the acidic protons with one, two or three cations. A person skilled in the art would understand that vanadium can form a coordination bond with at least one of the oxygen atoms in the OH groups of orthophosphoric acid.

The term “pharmaceutical composition of the application” or “pharmaceutical composition of the present application” and the like as used herein refers to a pharmaceutical composition comprising one or more vanadium compounds of the application.

The term “suitable” as used herein means that the selection of the particular composition or conditions would depend on the specific steps to be performed, the identity of the components to be transformed and/or the specific use for the compositions, but the selection would be well within the skill of a person trained in the art.

The present description refers to a number of chemical terms and abbreviations used by those skilled in the art. Nevertheless, definitions of selected terms are provided for clarity and consistency.

The term “cell” as used herein refers to a single cell or a plurality of cells and includes a cell either in a cell culture or in a subject.

The term “subject” as used herein includes all members of the animal kingdom including mammals, and suitably refers to humans. Thus the methods and uses of the present application are applicable to both human therapy and veterinary applications.

The term “pharmaceutically acceptable” means compatible with the treatment of subjects.

The term “pharmaceutically acceptable carrier” means a non-toxic solvent, dispersant, excipient, adjuvant or other material which is mixed with an active ingredient in order to permit the formation of a pharmaceutical composition, i.e., a dosage form capable of administration to a subject.

The term “solvate” as used herein means a compound, or a salt and/or prodrug of a compound, wherein molecules of a suitable solvent are incorporated in the crystal lattice. A suitable solvent is physiologically tolerable at the dosage administered.

The term “treating” or “treatment” as used herein and as is well understood in the art, means an approach for obtaining beneficial or desired results, including clinical results. Beneficial or desired clinical results can include, but are not limited to alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. “Treating” and “treatment” can also mean prolonging survival as compared to expected survival if not receiving treatment. “Treating” and “treatment” as used herein also include prophylactic treatment. For example, a subject with early cancer can be treated to prevent progression, or alternatively a subject in remission can be treated with a compound or composition of the application to prevent recurrence. Treatment methods comprise administering to a subject a therapeutically effective amount of one or more of the compounds of the application and optionally consist of a single administration, or alternatively comprise a series of administrations.

“Palliating” a disease or disorder means that the extent and/or undesirable clinical manifestations of a disorder or a disease state are lessened and/or time course of the progression is slowed or lengthened, as compared to not treating the disorder.

The term “prevention” or “prophylaxis”, or synonym thereto, as used herein refers to a reduction in the risk or probability of a patient becoming afflicted with a disease, disorder or condition, or manifesting a symptom associated with a disease, disorder or condition.

The term “administered” as used herein means administration of a therapeutically effective amount of a compound, or one or more compounds, or a composition of the application to a cell either in cell culture or in a subject.

As used herein, the term “effective amount” or “therapeutically effective amount” means an amount of a compound, or one or more compounds, that is effective, at dosages and for periods of time necessary to achieve the desired result.

The term “cancer” as used herein refers to cellular-proliferative disease states.

The term “increase” or “increasing” as used herein refers to any detectable increase or enhancement in a function or characteristic in the presence of one or more test variables, compared to otherwise the same conditions except in the absence of the one or more test variables.

The term “decrease” or “decreasing” as used herein refers to any detectable decrease or reduction in a function or characteristic in the presence of one or more test variables, compared to otherwise the same conditions except in the absence of the one or more test variables.

The term “oncolytic virus” as used herein refers to a virus that preferentially infects and lyses cancer or tumor cells as compared to non-cancer or normal cells.

The term “RNA virus” as used herein refers to a virus that has RNA (ribonucleic acid) as its genetic material. The nucleic acid can be either single-stranded RNA or double-stranded RNA, and the RNA can be either negative-sense or positive-sense.

By a “derivative” or “variant” of a virus as used herein, it is meant a virus obtained by selecting the virus under different growth conditions, one that has been subjected to a range of selection pressures, that has been genetically modified using recombinant techniques known within the art, or one that has been engineered to be replication defective and/or express transgenes, or any combination thereof.

### II. Compounds and Compositions of the Application

The present application includes a pharmaceutical composition comprising one or more vanadium compounds wherein the one or more vanadium compounds are selected from a citrate salt of vanadium, a phosphate salt of vanadium or mixed salts thereof.

In some embodiments, the one or more vanadium compounds comprise vanadium(IV). In some embodiments, the one or more vanadium compounds comprise vanadium(V).

In some embodiments, the citrate and/or phosphate salts of vanadium comprise one or more pharmaceutically acceptable solvent molecules within their structure, accordingly the salts are in the form of solvates. In some embodiments, the solvent is water, accordingly the salts are in the form of hydrates. In some embodiments, the vanadium salt comprises a mixture of citrate and phosphate salts.

In some embodiments, the one or more vanadium compounds are selected from the following structures, which are based on the interpretation of spectroscopic studies of solutions of material:

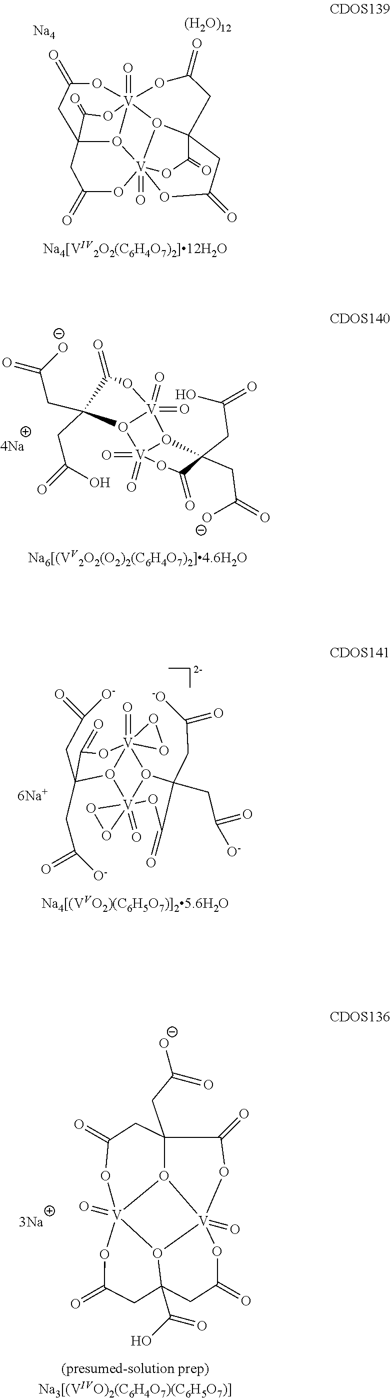

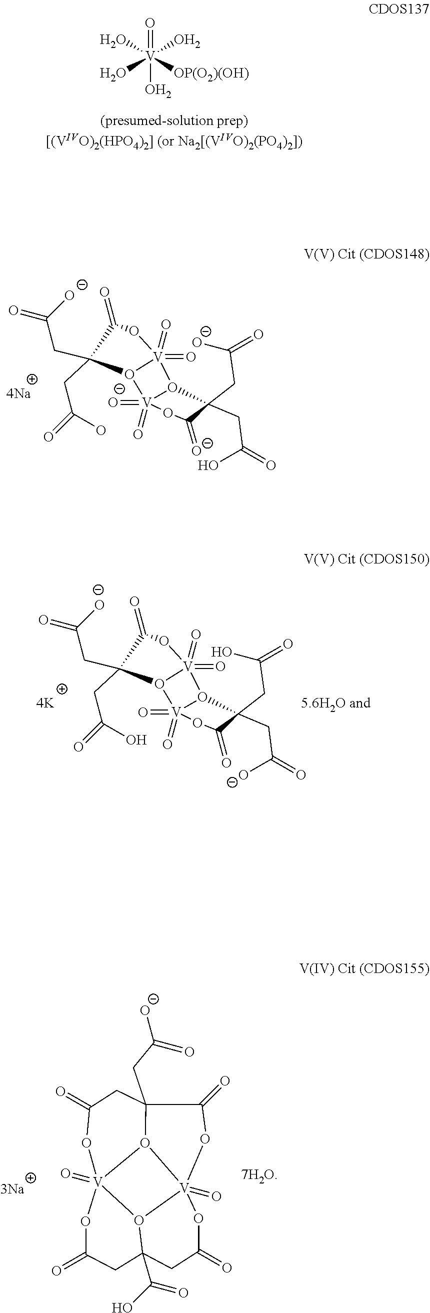

In some embodiments, the pharmaceutical composition further comprises one or more pharmaceutically acceptable carriers and/or excipients.

In some embodiments, the pharmaceutical composition further comprises one or more viruses selected from a therapeutic virus and a prophylactic virus. In some embodiments, the one or more viruses are selected from a non-naturally occurring DNA virus and a non-naturally occurring RNA virus. In some embodiments the one or more viruses are a genetically modified RNA virus, an attenuated RNA virus, or an oncolytic RNA virus, or a mixture thereof.

In some embodiments, the virus is an oncolytic RNA virus. In some embodiments, the oncolytic RNA virus is any suitable oncolytic RNA virus known in the art which infects and lyses cancer or tumor cells as compared to non-cancer or normal cells. For example, in some embodiments, the oncolytic virus is reovirus, newcastle disease virus, adenovirus, herpes virus, polio virus, mumps virus, measles virus, influenza virus, vaccinia virus, and/or rhabdoviruses such as vesicular stomatitis virus and derivatives/variants of each of the above. Examples of oncolytic viruses, and variants or derivatives thereof, are known in the art, for example from U.S. patent application publication nos. 20040115170, 20040170607, and 20020037543; PCT patent application publication no. WO 00/62735; and U.S. Pat. Nos. 7,052,832, 7,063,835, and 7,122,182 (which are each hereby incorporated by reference) and others.

In some embodiments, the virus is a vesicular stomatitis virus (VSV), or a related rhabdovirus variant/derivative thereof, for example, selected under specific growth conditions, one that has been subjected to a range of selection pressures, one that has been genetically modified using recombinant techniques known within the art, or a combination thereof. In some embodiments, the virus is VSVD51. Other derivatives or variants may be based on viruses such as Maraba (MG-1, for example), Farmington virus, rabies, Newcastle disease virus, poliovirus, zika virus, coronavirus, Coxsackie virus, semliki forest virus, ebolavirus, rift valley fever virus, Sindbis virus, Vaccinia virus, Herpes Simplex Virus, Poliovirus, Parvovirus, rotavirus, influenza, hepatitis A, mumps, measles, rubella, reovirus, dengue virus, Chikungunya virus, respiratory syncitial virus, LCMV, lentivirus, or replicating retrovirus, for example, and this is well within the purview of a person skilled in the art.

In some embodiments the pharmaceutical composition comprises a vanadium compound, an oncolytic virus and a pharmaceutically acceptable carrier and/or excipient, wherein the vanadium compound is a citrate salt of vanadium or a phosphate salt of vanadium.

In some embodiments, the amount of the one or more vanadium compounds in the pharmaceutical compositions of the application is that amount that increases the infection, spread, titer, activity, cytotoxicity and/or immunotherapeutic activity the one or more viruses in the pharmaceutical composition.

In some embodiments, the amount of the one or more vanadium compounds in the pharmaceutical compositions of the application is about 1 mg/mL to about 200 mg/mL, about 1 mg/mL to about 100 mg/mL, about 5 mg/mL to about 50 mg/mL, about 10 mg/mL to about 40 mg/mL, about 15 mg/mL to about 30 mg/mL, or about 20 mg/mL

In some embodiments, the one or more viruses are present in the pharmaceutical composition in therapeutically effective amounts. In some embodiments, the one or more viruses are present in the pharmaceutical composition in therapeutically effective amounts to treat cancer or a tumor.

In some embodiments, the amount of the one or more viruses in the pharmaceutical compositions of the application is about 2.5E9 pfu/ml, or about 2.5E5 pfu/ml to about 2.5E12 pfu/ml.

In some embodiments, the pH of the pharmaceutical compositions of the application is about 6 to about 9. In some embodiments, the pH of the pharmaceutical compositions of the application is about 6.5 to about 8.5. In some embodiments, the pH of the pharmaceutical compositions of the application is about 7 to about 8.

In some embodiments, the pharmaceutical composition comprises, one or more vanadium compounds and the one or more viruses in one or more pharmaceutically acceptable carriers and/or excipients. In some embodiments, the one or more carriers and/or excipients is water and optionally containing other solutes such as dissolved salts and the like. In some embodiments, the solution comprises enough saline, glucose or the like to make the solution isotonic. Pharmaceutical compositions and methods of preparing pharmaceutical compositions are known in the art and are described, for example, in “Remington: The Science and Practice of Pharmacy” (formerly “Remingtons Pharmaceutical Sciences”); Gennaro, A., Lippincott, Williams & Wilkins, Philidelphia, PA (2000), herein incorporated by reference.

In some embodiments, the pharmaceutical composition is for administration by injection. In some embodiments, the pharmaceutical composition is for administration by intravenous or intratumor injection. Pharmaceutical compositions suitable for injection include, for example, sterile aqueous solutions or dispersions and sterile powders for the extemporaneous preparation of sterile injectable solutions or dispersions. In all cases, the composition for pharmaceutical use must be sterile and must be fluid to the extent that easy syringability exists.

The compounds and pharmaceutical compositions of the application may be administered to a subject in a variety of forms depending on the selected route of administration, as will be understood by those skilled in the art. A vanadium compound or composition of the application may be administered, for example, by oral, parenteral, buccal, sublingual, nasal, rectal, patch, pump or transdermal administration, or injection such as intravenous, intratumoral injection, subcutaneous injection, intraperitoneal injection, bladder or other instillation, and the pharmaceutical compositions formulated accordingly. Administration can be by means of a pump for periodic or continuous delivery. Conventional procedures and ingredients for the selection and preparation of suitable compositions are described, for example, in Remington's Pharmaceutical Sciences (2000-20th edition) and in The United States Pharmacopeia: The National Formulary (USP 24 NF19) published in 1999.

The present application further includes a kit comprising: one or more vanadium compounds of the present application; and one or more viruses.

The present application further includes a kit comprising: a pharmaceutical composition of the present application.

In some embodiments, additional components are included in the kits of the present application, such as one or more pharmaceutically acceptable carriers, excipients, administration means (e.g. syringes), and/or instructions for use. Selection of additional suitable components is well within the purview of a person skilled in the art.

In some embodiments, the pharmaceutical compositions of the present application further comprise one or more additional therapeutic agents, for example one or more anticancer agents known in the art.

### III. Methods and Uses of the Application

The compounds and compositions of the application have been shown to be useful for increasing the infection, spread, titer, oncolytic activity, cytotoxicity and/or immunotherapeutic activity of an oncolytic RNA virus. Therefore the compounds and compositions of the application may be useful in compositions for the treatment of cancer or tumors,

Accordingly, the present application includes a use of one or more vanadium compounds of the application for increasing the infection, spread, titer, activity, cytotoxicity and/or immunotherapeutic activity of a virus.

In some embodiments, the present application includes a use of one or more vanadium compounds of the application for increasing activity of a virus.

In some embodiments, the present application includes a use of one or more vanadium compounds of the application for increasing immunotherapeutic activity of a virus.

In some embodiments, the present application includes a use of one or more vanadium compounds of the application for increasing cytotoxicity of a virus in cells.

The present application also includes a use of one or more vanadium compounds of the application in the manufacture of a medicament for increasing the infection, spread, titer, activity, cytotoxicity and/or immunotherapeutic activity of a virus.

In some embodiments, the present application includes a use of one or more vanadium compounds of the application in the manufacture of a medicament for increasing oncolytic activity of an oncolytic RNA virus in cancer or tumor cells.

In some embodiments, the present application includes a use of one or more vanadium compounds of the application in the manufacture of a medicament for increasing immunotherapeutic activity of a virus.

In some embodiments, the present application also includes a use of one or more vanadium compounds of the application in the manufacture of a medicament for increasing cytotoxicity of a virus in cells.

In some embodiments, the present application includes a use of one or more vanadium compounds of the application in combination with one or more viruses for increasing permissiveness of cells to one or more viruses. In some embodiments, the present application includes a use of one or more vanadium compounds of the application in combination with one or more viruses for preparation of a medicament for increasing permissiveness of cells to one or more viruses. In some embodiments, increasing the permissiveness of a cell to a virus relates to a decrease of the cell's immune response to the virus, for example type I interferon immune response, such that infection, spread, titer, activity, transduction, cytotoxicity and/or immunotherapeutic activity of the virus, or genetic components encoding the same, in the cell is increased. By decreasing a cell's immune response to a virus, for example for example type I interferon immune response, a person skilled in the art would appreciate that the virus will be permitted to survive, grow and/or spread in the cell and thereby increasing the virus' infection, spread, titer, activity, cytotoxicity and/or immunotherapeutic activity. A skilled person in the art would understand that permissiveness of a cell to a virus correspond to viral sensitization, as used herein.

Also included in the present application is a method of increasing infection, spread, titer, activity, cytotoxicity and/or immunotherapeutic activity of one or more viruses, comprising administering, to cells or a subject in need thereof, an effective amount of one or more vanadium compounds of the application in combination with the one or more viruses.

In some embodiments, the present application includes a method of increasing activity of one or more viruses, comprising administering, to cells or a subject in need thereof, an effective amount of one or more vanadium compounds of the application in combination with the one or more viruses.

In some embodiments, the present application includes a method of increasing immunotherapeutic activity of one or more viruses, comprising administering, to cells or a subject in need thereof, an effective amount of one or more vanadium compounds of the application in combination with the one or more viruses.

In some embodiments, the present application includes a method of increasing cytotoxicity of one of more viruses, comprising administering, to cells or a subject in need thereof, an effective amount of one or more vanadium compounds of the application in combination with the one or more viruses.

In some embodiments, the present application includes a method for increasing permissiveness of cells to one or more viruses, comprising administering to the cells an effective amount of one or more vanadium compounds of the application in combination with an effective amount of the one or more viruses.

In some embodiments, the one or more viruses are administered to the cell for production of the one or more viruses by the cell and the one or more vanadium compounds increase permissiveness of the cell to the one or more viruses to increase the amount of virus produced by the cell.

In some embodiments, the one or more viruses are comprised a non-replicating viral vector. In some embodiments, the viral vector is an adenovirus (Ad), an adeno-associated virus (AAV) or lentivirus (LV).

In some embodiments, the viral vector is administered to the cell for transduction of the cell and the one or more vanadium compounds increase permissiveness of the cell to the viral vector to increase the amount of viral transduction.

In some embodiments, the viral vector comprises a therapeutic gene and one or more vanadium compounds increase permissiveness of the cell to the viral vector to increase the amount of virally-encoded therapeutic gene incorporated into the genome of the cell.

In some embodiments, the one or more viruses are administered to the cell to treat a disease, disorder or condition in the cell and the one or more vanadium compounds increase permissiveness of the cell to the one ore more viruses to increase the efficacy of the for treating the disease, disorder or condition.

In some embodiments, transduction, infection and/or growth of the one or more viruses in the cell is increased.

In some embodiments, the virus is an oncolytic virus and the cells are cancer or tumor cells and the subject has cancer or a tumor.

In some embodiments, the virus is an oncolytic RNA virus. In some embodiments, the oncolytic RNA virus is any suitable oncolytic RNA virus known in the art which infects and lyses cancer or tumor cells as compared to non-cancer or normal cells. For example, in some embodiments, the oncolytic virus is reovirus, newcastle disease virus, adenovirus, herpes virus, polio virus, mumps virus, measles virus, influenza virus, vaccinia virus, and/or rhabdoviruses such as vesicular stomatitis virus and derivatives/variants of each of the above. Examples of oncolytic viruses, and variants or derivatives thereof, are known in the art, for example from U.S. patent application publication nos. 20040115170, 20040170607, and 20020037543; PCT patent application publication no. WO 00/62735; and U.S. Pat. Nos. 7,052,832, 7,063,835, and 7,122,182 (which are each hereby incorporated by reference) and others.

In some embodiments, the virus is a vesicular stomatitis virus (VSV), or a related rhabdovirus variant/derivative thereof, for example, selected under specific growth conditions, one that has been subjected to a range of selection pressures, one that has been genetically modified using recombinant techniques known within the art, or a combination thereof. In some embodiments, the virus is VSVΔ51. Other derivatives or variants may be based on viruses such as Maraba (MG-1, for example), rabies, rotavirus, influenza, hepatitis A, mumps, measles, rubella, reovirus, dengue virus, Chikungunya virus, respiratory syncitial virus, LCMV, lentivirus, or replicating retrovirus, for example, and this is well within the purview of a person skilled in the art.

In some embodiments, the cancer or tumor is lymphoblastic leukemia, myeloid leukemia, adrenocortical carcinoma, AIDS-related cancer, AIDS-related lymphoma, anal cancer, appendix cancer, astrocytoma, atypical teratoid/rhabdoid tumor, basal cell carcinoma, bile duct cancer, bladder cancer, bone cancer, osteosarcoma, malignant fibrous histiocytoma, brain stem glioma, brain tumor, cerebellar astrocytoma, cerebral astrocytoma/malignant glioma, craniopharyngioma, ependymoblastoma, medulloblastoma, pineal parenchymal tumors of intermediate differentiation, supratentorial primitive neuroectodermal tumors and pineoblastoma, visual pathway and hypothalamic glioma, spinal cord tumors, breast cancer, bronchial tumors, Burkitt lymphoma, carcinoid tumor, central nervous system lymphoma, cervical cancer, chordoma, chronic lymphocytic leukemia, chronic myelogenous leukemia, chronic myeloproliferative disorders, colon cancer, cutaneous T-Cell lymphoma, embryonal tumors, endometrial cancer, ependymoblastoma, ependymoma, esophageal cancer, extracranial germ cell tumor, extragonadal germ cell tumor, extrahepatic bile duct cancer, eye cancer, intraocular melanoma, retinoblastoma, gallbladder cancer, gastric (stomach) cancer, gastrointestinal carcinoid tumor, gastrointestinal stromal tumor (GIST), gastrointestinal stromal cell tumor, germ cell tumors, extracranial, extragonadal, ovarian, gestational trophoblastic tumor, glioma, hairy cell leukemia, head and neck cancer, hepatocellular (liver) cancer, histiocytosis, Langerhans cell cancer, Hodgkin lymphoma, hypopharyngeal cancer, islet cell tumors, Kaposi sarcoma, kidney cancer, laryngeal cancer, lymphocytic leukemia, hairy cell leukemia, lip and oral cavity cancer, liver cancer, non-small cell lung cancer, small cell lung cancer, Hodgkin lymphoma, non-Hodgkin lymphoma, malignant fibrous histiocytoma of bone and osteosarcoma, medulloblastoma, medulloepithelioma, melanoma, intraocular melanoma, Merkel cell carcinoma, mesothelioma, metastatic squamous neck cancer, mouth cancer, multiple endocrine neoplasia syndrome, multiple myeloma/plasma cell neoplasm, nasal cavity and paranasal sinus cancer, nasopharyngeal cancer, neuroblastoma, oral cancer, oropharyngeal cancer, ovarian cancer, pancreatic cancer, parathyroid cancer, penile cancer, pharyngeal cancer, pheochromocytoma, pineal parenchymal tumors, pineoblastoma and supratentorial primitive neuroectodermal tumors, pituitary tumor, plasma cell neoplasm/multiple myeloma, pleuropulmonary blastoma, primary central nervous system lymphoma, prostate cancer, rectal cancer, renal cell (kidney) cancer, renal pelvis and ureter cancer, transitional cell cancer, respiratory tract carcinoma, retinoblastoma, rhabdomyosarcoma, salivary gland cancer, uterine sarcoma, skin cancer, Merkel cell skin carcinoma, small intestine cancer, soft tissue sarcoma, squamous cell carcinoma, squamous neck cancer, stomach (gastric) cancer, supratentorial primitive neuroectodermal tumors, T-Cell lymphoma, testicular cancer, throat cancer, thymoma and thymic carcinoma, thyroid cancer, trophoblastic tumor, urethral cancer, uterine cancer, endometrial cancer, uterine sarcoma, vaginal cancer, vulvar cancer, and/or Wilms tumor.

In some embodiments, the present application includes a use of one or more vanadium compounds of the application in combination with one or more oncolytic viruses for the treatment of a cancer or a tumor in a subject or cell in need thereof. In some embodiments, the present application includes a use of one or more vanadium compounds of the application in combination with one or more oncolytic viruses for the preparation of a medicament for treatment of a cancer or a tumor in a subject or cell in need thereof. The present application also includes a method of treating a cancer or a tumor, comprising administering an effective amount of one or more vanadium compounds of the application in combination with an effective amount of one or more oncolytic viruses to a subject or cell in need thereof.

In some embodiments the one or more vanadium compounds of the application are used or administered to the cancer cells, tumor cells, or subject in need thereof prior to, concurrently with or after the one or more oncolytic viruses are used or administered to the cancer cells, tumor cells or subject in need thereof.

In some embodiments, the one or more vanadium compound is comprised in a pharmaceutical composition of the application therefore the one or more vanadium compounds of the application are used or administered to the cancer cells, tumor cells, or subject in need concurrently with the one or more oncolytic viruses.

In some embodiments, the cancer cells or tumor cells are in vitro or in vivo.

In some embodiments, the subject is a mammalian subject. In some embodiments, the subject is a human subject.

In the context of cancer or a tumor in a subject, an effective amount is an amount that, for example, treats the cancer or tumor, compared to the treatment without administration of the one or more vanadium compounds of the application in combination with the one or more oncolytic viruses or the compositions of the application. Effective amounts may vary according to factors such as the disease state, age, sex and/or weight of the subject. The amount of a given vanadium compound and/or virus that will correspond to such an amount will vary depending upon various factors, such as the given compound and/or virus, the pharmaceutical formulation, the route of administration, the type of cancer or tumor, the identity of the subject being treated, and the like, but can nevertheless be routinely determined by one skilled in the art. The effective amounts are ones that, following treatment therewith, manifests as an improvement in or reduction of any disease symptom. When the disease is cancer or tumors, amounts that are effective can cause a reduction in the number, growth rate, size and/or distribution of the cancer or tumors.

In some embodiments, the one or more vanadium compounds of the application in combination with the one or more oncolytic viruses or the compositions of the application are administered or used in a single daily, weekly or monthly dose or the total daily dose may be divided into two, three or four daily doses. The length of the treatment period depends on a variety of factors, such as the severity of the disease, the age of the subject, the concentration and/or the activity of the vanadium compounds of the application, the concentration and/or the activity of the one or more viruses, or a combination thereof. It will also be appreciated that effective amounts used for treatment may increase or decrease over the course of a particular treatment regime. Changes in amounts or dosages may result and become apparent by standard diagnostic assays known in the art. In some instances, chronic administration is required.

In some embodiments, in any methods of the application, the one or more vanadium compounds of the application may be administered in an amount of about 10 mg/kg to about 200 mg/kg, and the one or more virus may be administered in an amount of about 1E6-1E9 pfu per dose. A skilled person in the art would understand that dosing may change significantly depending on the application and type of virus, and would appreciate how to adjust accordingly.

The present application includes a use of one or more vanadium compounds of the application as an adjuvant for a virus-based vaccine, such as an RNA virus-based cancer vaccine.

The present application also includes a use of one or more vanadium compounds of the application as an adjuvant in the manufacture of a virus-based vaccine, such as an RNA virus-based cancer vaccine.

The present application also includes a method or preparing a virus-based vaccine comprising combining one or more vanadium compounds of the application with one or more viruses under conditions to prepare a vaccine. In some embodiments, the one or more viruses are oncolytic viruses, such as oncolytic RNA virus, and the vaccine is a cancer vaccine.

### IV. Methods of Preparing the Compounds and Compositions of the Application

The vanadium compounds of the present application, and composition comprising the same, may be obtained from known methods in the art.

In some embodiments, the vanadium compounds are prepared using methods described by Salifoglou, A. (2001) Inorganic Chemistry, 40(23), 5772-5779; Kaliva, Inorg. Chem. 2002, 41, 3850-3858; Kaliva et al. Inorganic Chemistry 2004, 43, 2895-2905. A skilled person in the art would appreciate that other methods can be used. As a result, different precursors are contemplated. For example, for the vanadium(IV) complexes, both VCl₃ and VOSO₄ can be used. For the vanadium(V) both V₂O₅, and simple metavanadate or orthovanadate compounds could be used as precursors, such as NaVO₃ or Na₃VO₄ or other similar salts.

### EXAMPLES

The following non-limiting examples are illustrative of the present application.

### General Methods

The vanadium compounds were obtained from preparations of soluble materials (100 mM concentration of V-atom) such as CDOS136 [579.89 g/mol] and CDOS137 [325.75 g/mol] or from solid material (CDOS139 [817.94 g/mol], CDOS140 [635.87 g/mol], CDOS141[711.82 g/mol]). CDOS139, was prepared using the method previously described by Salifoglou, based on VCl₃ precursor, which contains V in oxidation state III. CDOS140 is a Na+ salt and was prepared using a method previously reported by Kaliva 2002, from V₂O₅ as the K⁺ salt. Hence counter ions can be changed and identical V-citrate-anions in materials with different counterions. CDOS141 was prepared using the method reported by Kaliva 2004, using V₂O₅. This was reported to be prepared from both VCl₃ and V₂O₅, dissolving the vanadium precursor in water and adding the citric acid/citrate precursor. In addition to reported procedures, other procedures can be used for preparation of both the vanadium(IV) citrate complexes (CDOS136, CDOS139), the vanadium(V) citrate complexes (CDOS140, CDOS141) and the vanadium(IV) phosphate complex (CDOS137). Compound composition and molecular structures/weight were confirmed by routine spectroscopic methods and/or elemental analysis.

### Results

### Example 1

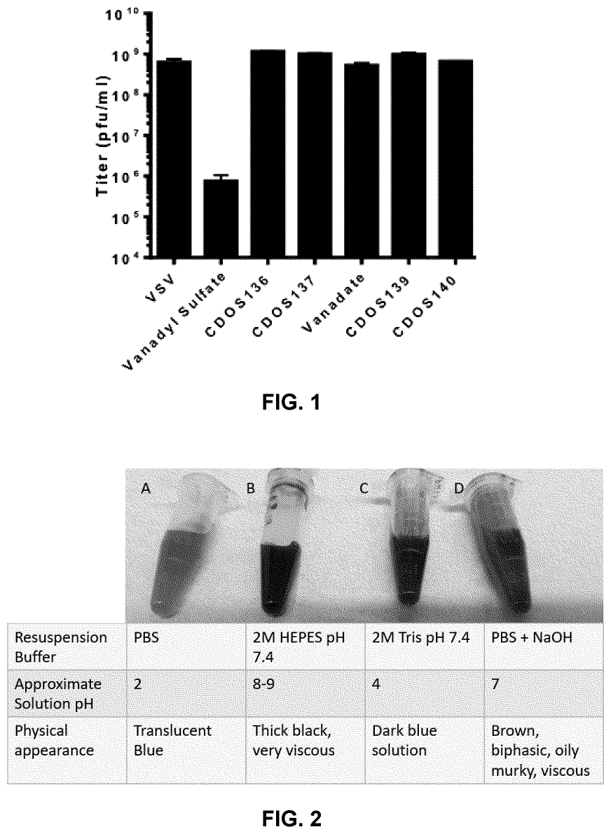

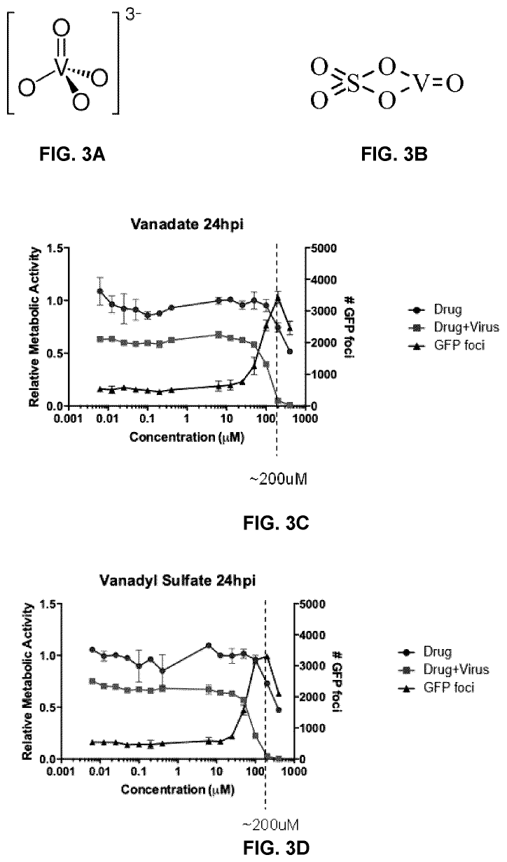

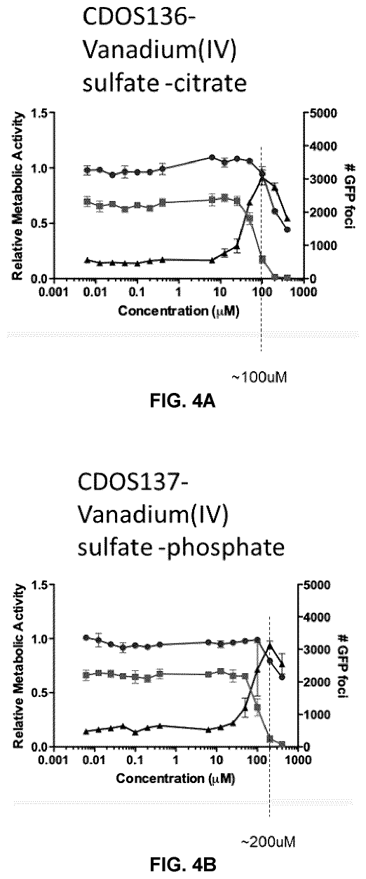

Because most viruses are sensitive to alterations in pH, vanadyl sulfate has significant shortcomings for co-formulation with therapeutic viruses. For example, the oncolytic virus vesicular stomatitis virus (VSV) is highly sensitive to pH below 6 and degrades rapidly in an acidic environment. This problem is not unique to VSV as many other viruses, for example but not limited to measles (“Forced Degradation Studies to Identify Critical Process Parameters for the Purification of Infectious Measles Virus”), cocksackievirus, reovirus, coronavirus, and influenza virus, are pH sensitive. Importantly, unbuffered vanadyl sulfate at the required concentration to enhance oncolytic VSV in vivo is highly acidic and leads to rapid inactivation of VSV when admixed in co-formulation (see FIG. 1). Adjustment towards more neutral pH using NaOH in PBS or using other buffered solutions such as 2M TRIS or HEPES is either insufficient to achieve pH>6 or leads to unacceptable changes in physicochemical properties such as increased viscosity and phase separation (FIG. 2). To overcome the limitations of vanadyl sulfate for co-formulation with viruses, different formulations of vanadium were tested and their viral sensitization effects compared to standard solutions of vanadate and vanadyl sulfate which are illustrated in FIG. 3. FIG. 3 shows that in 786-0 renal carcinoma cells vanadate and vanadyl sulfate both lead to a cytotoxicity (relative metabolic activity) differential in presence and absence of VSVD51 which is associated with increased oncolytic activity in presence of the compounds. An accompanying increase in GFP foci, associated with increased spread of the virus which here encodes GFP, is also observed. As a starting point, vanadyl sulfate solutions brought to neutral pH by using either biologically compatible citrate (CDOS136) or phosphate (CDOS137) were made and tested. FIG. 4 shows that CDOS136 and CDOS137 provided similarly beneficial impact on oncolytic VSVD51 spread in 786-0 human renal cancer cells. In comparison to vanadyl sulfate, the dose at which peak viral sensitization activity was observed was either similar or improved in the case of citrate buffer (approximately 200 uM for vanadyl sulfate compared to 100 uM for CDOS136).

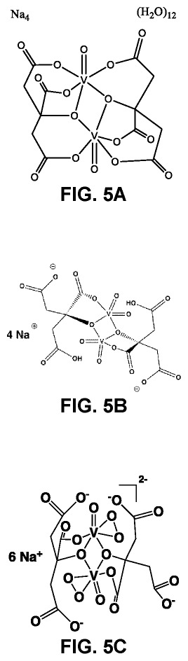

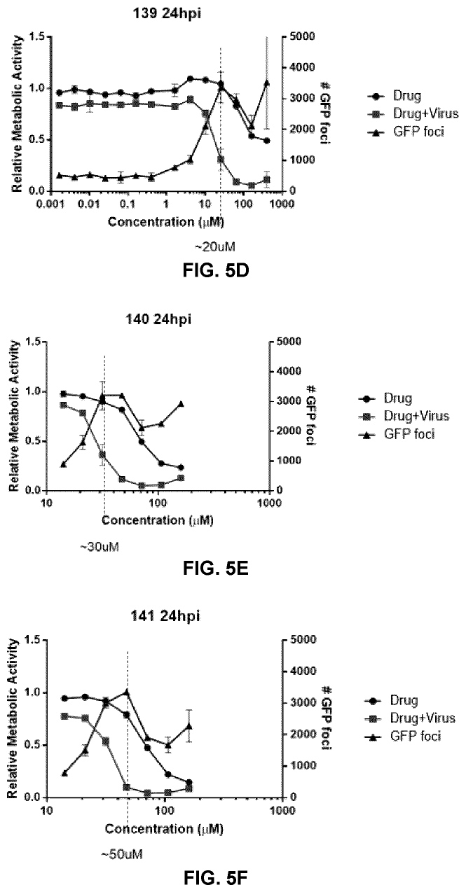

Based on the potency gain with CDOS136, vanadium (IV) citrate (CDOS139), vanadium (V) citrate (CDOS140), or vanadium (V) citrate peroxo (CDOS141), which naturally form solutions at an appropriately neutral pH, were synthesized. FIG. 5 and Table 1 below show that each compound exhibited viral sensitization activity, but potency was substantially improved over vanadyl sulfate with the most potent compound CDOS139 showing peak activity at approximately 20 uM.

TABLE 1
Summary of Viral Sensitizing Impact of Vanadium compounds.

| | Approximate Peak Viral |
|---|---|
| Compound | Sensitizer Dose (uM) |
| Vanadate | 200 |
| Vanadyl sulfate | 200 |
| CDOS136 | 100 |
| CDOS137 | 200 |
| CDOS139 | 20 |
| CDOS140 | 30 |
| CDOS141 | 50 |

A summary of the concentration at which peak enhancement in VSVD51-GFP growth (GFP foci) is obtained for the compounds is presented in FIGS. 2-5.

The capacity of these different compounds to be admixed with VSVD51 for a period of 1 h was next tested. Replicating anticipated dosing in animals, virus and compounds (20 ul of 40 mg/ml vanadium, 20 ul of 5E9 pfu/ml VSVD51 for approximately 40 mg/kg+1E8 PFU/dose) or a mock control were combined and viral titers assessed after 1 h incubation at room temperature to indicate whether virus was stable for one or more hour after admixing. FIG. 5 shows that CDOS136, CDOS137, CDOS139, CDOS140 and vanadate, led to dramatically better stability of VSVD51 over 1 h as compared to vanadyl sulfate, wherein viral titers recovered were improved or similar to the control compared to >1000-fold decrease in viral titers with vanadyl sulfate. Taken together the data in FIGS. 1-5 suggest that CDOS136, CDOS139, and CDOS140 in particular have an improved capacity for co-formulation with VSV because they are more potent than standard preparations of either vanadate or vanadyl sulfate and retain virus stability after admixing for a period of at least 1 h.

### Example 2

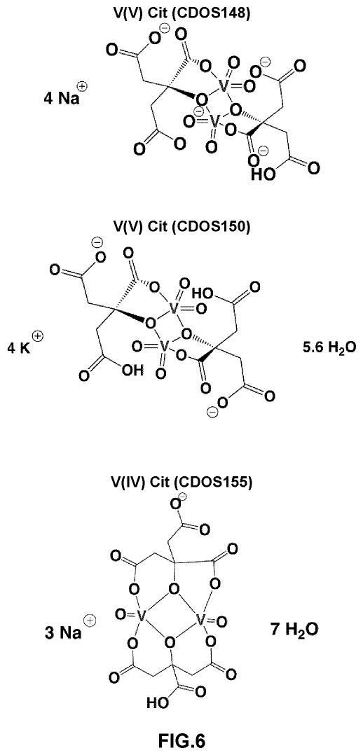

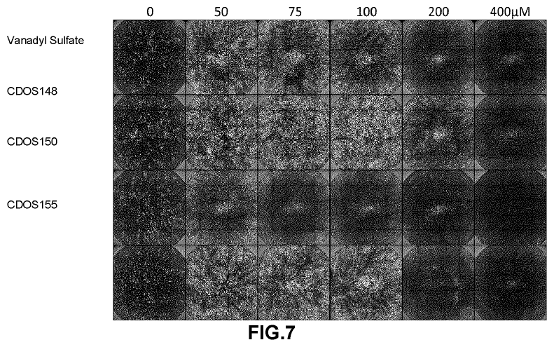

Other exemplary vanadium IV/V-citrate preparations (CDOS148, CDOS150, and CDOS155) were prepared and their structures derived from spectroscopic studies of solutions of material are shown in FIG. 6. CDOS148 dissolved naturally at a neutral pH in water, whereas CDOS150 and CDOS155 dissolved in water at pH 5.2 but can be adjusted to pH 7-8 using either 1.2M Citrate buffer or 1M NaOH without precipitation at concentrations of at least up to 50 mg/ml. In Table 2, these vanadium IV/V-citrate preparations at neutral pH were tested for their ability to enhance the growth of VSVd51-GFP in 786-0 renal carcinoma cells as measured by GFP expression, indicating viral growth spread. Table 2 reports the fold change in GFP-positive area (i.e. infected cells) at different concentrations of the V-(IV) and V-(V)-citrate compounds tested in comparison to vanadyl sulfate. The results show that these compounds all enhance VSV growth compared to the untreated control (Concentration 0.0), and that V(V) compounds show better potency compared to vanadyl sulfate, as defined by improved fold increases in virus spread at lower doses. In FIG. 7, a similar experiment was repeated but in mouse CT26 wt colon carcinoma cells, confirming the activity of these compounds in a different species and cancer type, as has been shown previously in a wide range of cancer types and species of origin for vanadyl sulfate and other vanadium compounds. These data altogether further support that various V(IV) and V(V)-citrate preparations are amenable to use at neutral pH and can effectively enhance virus growth in cancer cells originating from different tissues (kidney, colon) and different species (mouse, human).

TABLE 2
Fold change in GFP following VSVΔ51-
GFP infection in 786-0 cells

| | Vanadyl | | | |
|---|---|---|---|---|
| Concentration | Sulfate | CDOS150 | CDOS155 | CDOS148 |
| 0.0 | 1 | 1 | 1 | 1 |
| 35.1 | 11 | 12 | 7 | 12 |
| 52.7 | 24 | 41 | 19 | 34 |
| 79.0 | 34 | 51 | 22 | 43 |
| 88.0 | 43 | 51 | 25 | 87 |
| 118.5 | 79 | 55 | 47 | 148 |
| 132.0 | 89 | 62 | 55 | 120 |
| 177.8 | 119 | 62 | 51 | 93 |
| 198.0 | 114 | 70 | 62 | 174 |
| 266.7 | 156 | 76 | 86 | 141 |
| 296.0 | 178 | 81 | 83 | 131 |
| 400.0 | 200 | 76 | 121 | 78 |
| 444.0 | 141 | 86 | 129 | 113 |
| 667.0 | 119 | 68 | 130 | 49 |
| 1000.0 | 76 | 36 | 155 | 47 |

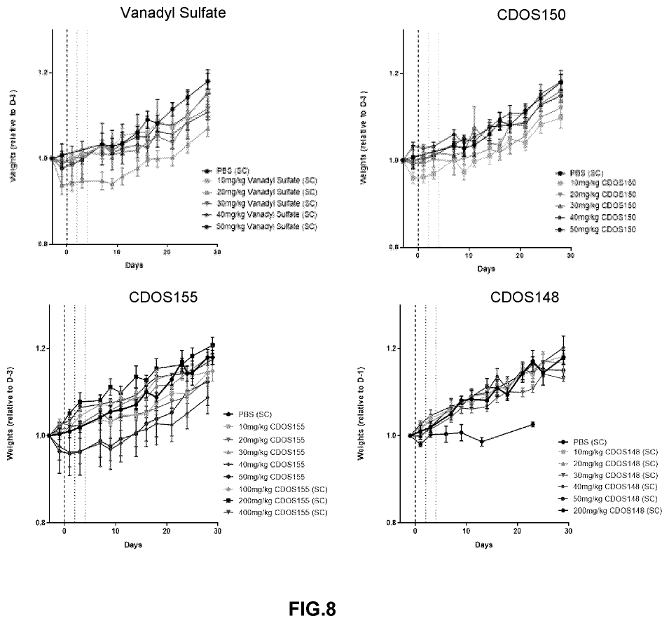

V(IV) and V(V)-citrate preparations CDOS148, CDOS150 and CDOS155 prepared at neutral pH (using Citrate buffer 1.2M for CDOS150 and CDOS155) were next administered to naive Balb/C mice at increasing doses. In Table 3, an estimated maximum tolerated dose for 3 repeated intraperiteoneal administrations, every other day, is reported. The estimated maximum tolerated dose being defined as the highest dose at which no mice required humane endpoint. FIG. 8 shows the normalized weights of mice over time at the different dosing levels. From these data, it emerges that the V(IV)-citrate compound CDOS155 is the best tolerated, with an estimated maximum tolerated dose of approximately 400 mg/kg, contrasting sharply with vanadyl sulfate, which is tolerated up to 50 mg/kg.

TABLE 3
Estimated maximum tolerated dose for 3 repeated
intraperiteoneal administrations

| Compound | Estimated Maximum tolerated dose |
|---|---|
| Vanadyl Sulfate | 50 mg/kg |
| CDOS148 (Vanadium(V)-Citrate) | 50 mg/kg |
| CDOS150 (Vanadium(V)-Citrate) | 50 mg/kg |
| CDOS155 (Vanadium(IV)-Citrate) | 400 mg/kg |

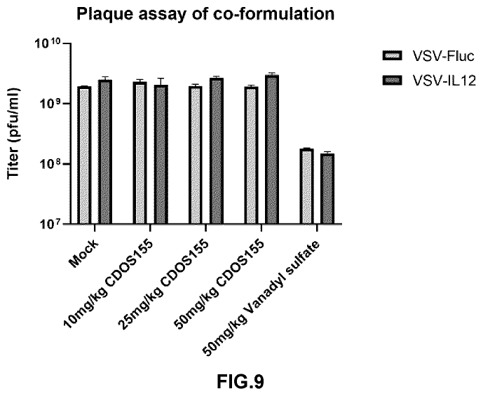

The ability of the best tolerated V-(IV)-citrate compound CDOS155 to be co-formulated with a virus was next tested. For this experiment, two different derivatives of VSVd51 were used: one encoding Firefly luciferase (VSV-FLuc) and the other encoding the IL12 pro-inflammatory cytokine (VSV-IL12). Consistent with other V-citrate and V-phosphate preparations, FIG. 9 shows that co-mixing the V(IV)-citrate compound CDOS155 used at neutral pH (neutralized with citrate Buffer 1.2M) with the above viruses at CDOS155 concentrations up to at least 50 mg/kg prior to virus titration does not impact the effective titer as measured by plaque assay of either VSV-Fluc or VSV-IL12, in sharp contrast with vanadyl sulfate.

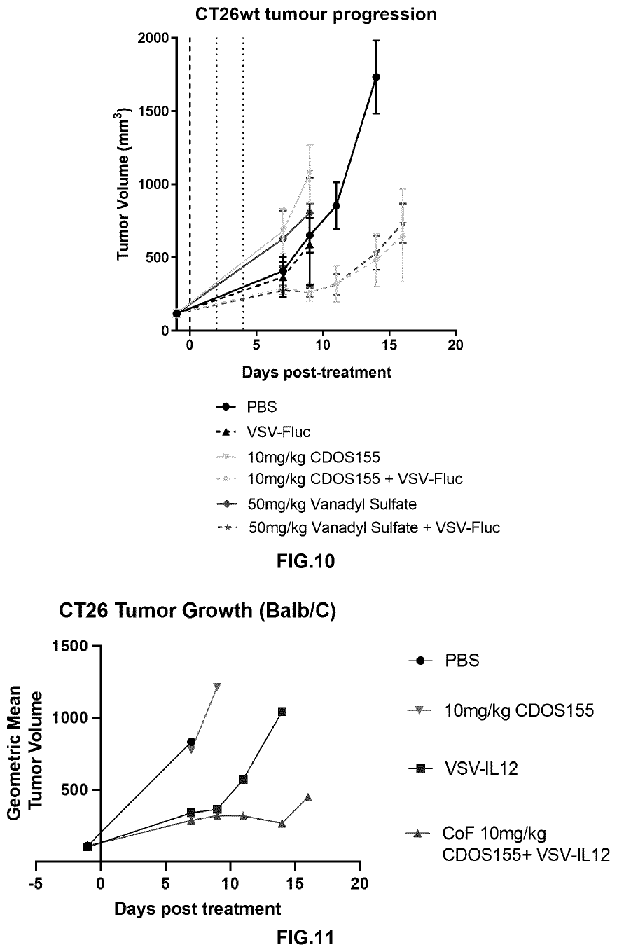

The V(IV)-Citrate compound CDOS155 used at neutral pH (citrate buffer) was next evaluated for its capacity to enhance the antitumor effects of VSV-Fluc. For these experiments, CT2 wt mouse colon cancer cells were implanted in syngeneic Balc/C mice. FIG. 10 shows that CDOS155 used at 10 mg/kg when administered separately (15 minutes apart) from VSV-Fluc (1E8 plaque forming units, PFU) intratumorally enhanced the antitumor effects of VSV-Fluc to a comparable extent to a similar regimen of vanadyl sulfate 50 mg/kg.

The same Ct26 wt syngeneic tumor model was used to evaluate to potential of CDOS155 to be co-formulated with VSV-IL12. A co-formulation of CDOS155 (neutral pH, 1.2M citrate buffer) was prepared with VSV-IL12 resulting in a single injectable co-formulated dose of 10 mg/kg CDOS155: 1E8 PFU VSV-IL12. As presented in FIG. 11, this co-formulation administered three times (days 0, 2, 4) led to delayed tumor progression compared to CDOS155 and VSV-IL12 alone. Altogether, this suggests that the V(IV)-citrate compound CDOS155 is well tolerated and effective as part of a co-formulation with oncolytic VSV, including but not limited to VSV-IL12.

While the applicant's teachings described herein are in conjunction with various embodiments for illustrative purposes, it is not intended that the applicant's teachings be limited to such embodiments as the embodiments described herein are intended to be examples. On the contrary, the applicant's teachings described and illustrated herein encompass various alternatives, modifications, and equivalents, without departing from the embodiments described herein, the general scope of which is defined in the appended claims.

1. A pharmaceutical composition comprising one or more vanadium compounds wherein the one or more vanadium compounds are selected from a citrate salt of vanadium, a phosphate salt of vanadium or mixed salts thereof.

2. The pharmaceutical composition of claim 1, wherein the one or more vanadium compounds comprise vanadium (IV) and/or vanadium (V).

3. (canceled)

4. The pharmaceutical composition of claim 1, wherein the one or more vanadium compounds are selected from:

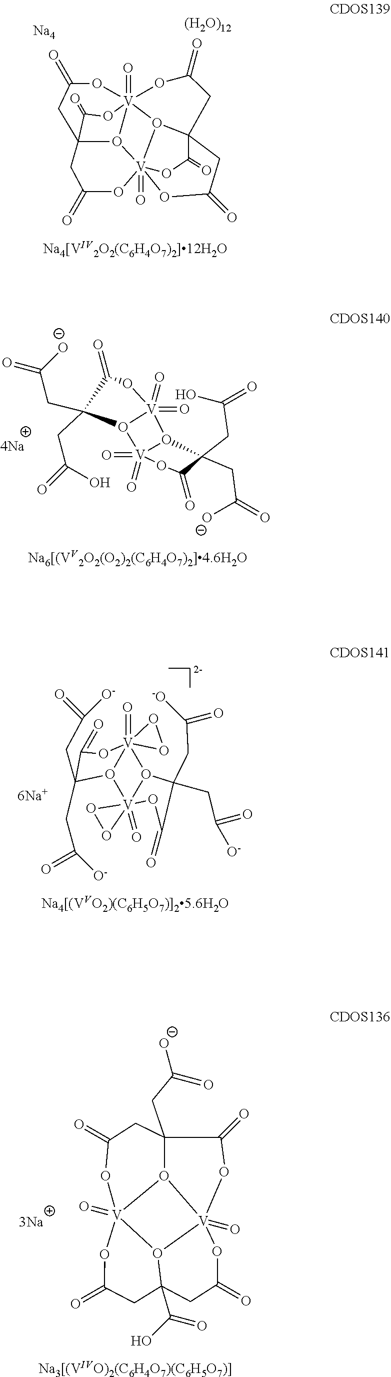

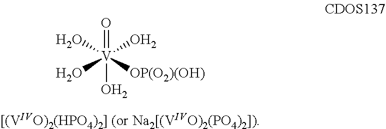

5. The pharmaceutical composition of claim 1, wherein the one or more vanadium compounds are selected from:

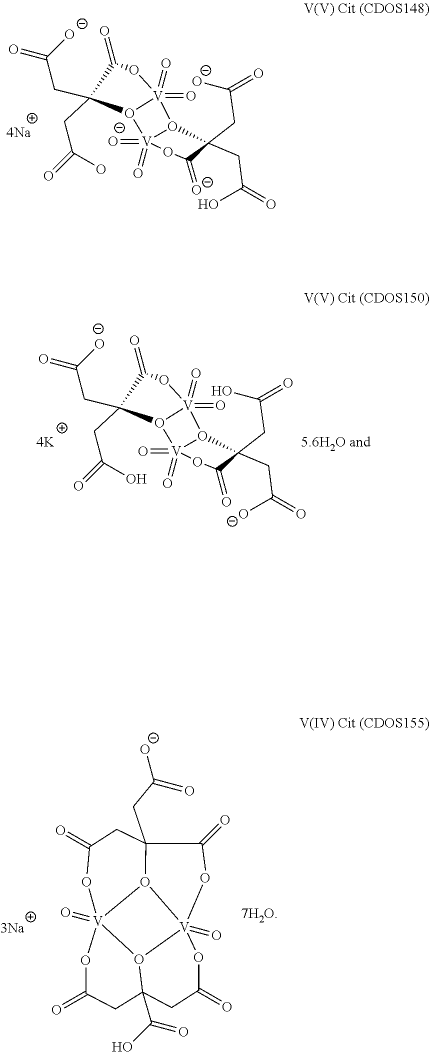

6. The pharmaceutical composition of claim 1, further comprising one or more pharmaceutically acceptable carriers and/or excipients.

7. The pharmaceutical composition of claim 1, further comprising one or more viruses.

8. The pharmaceutical composition of claim 7, wherein the one or more viruses are selected from a therapeutic virus and a prophylactic virus.

9. The pharmaceutical composition of claim 7, wherein the one or more viruses are selected from a non-naturally occurring DNA virus and a non-naturally occurring RNA virus.

10. The pharmaceutical composition of claim 7, wherein the one or more viruses are an oncolytic RNA virus.

11. The pharmaceutical composition of claim 7, wherein the one or more viruses are Newcastle Disease virus, Maraba virus, Farmington virus, poliovirus, measles virus, Coxsackie virus, reovirus, replicating retrovirus or a derivative or variant thereof.

12. The pharmaceutical composition of claim 7, wherein the one or more viruses are a vesicular stomatitis virus or a derivative or variant thereof.

13. (canceled)

14. (canceled)

15. (canceled)

16. (canceled)

17. (canceled)

18. (canceled)

19. A method of treating a cancer or tumor, comprising administering an effective amount of a pharmaceutical composition of claim 7 to a cell or subject in need thereof.

20. A method of increasing infection, spread, titer, activity, cytotoxicity and/or immunotherapeutic activity of one or more viruses, comprising administering, to cells or a subject in need thereof, an effective amount of one or more vanadium compounds as defined in claim 1 in combination with an effective amount of the one or more viruses.

21. A method for increasing permissiveness of cells to one or more viruses, comprising administering to the cells an effective amount of one or more vanadium compounds as defined in claim 1 in combination with an effective amount of the one or more viruses.

22. (canceled)

23. The method of claim 20, wherein the one or more viruses are administered to the cell for production of the one or more viruses by the cell and the one or more vanadium compounds increase permissiveness of the cell to the one or more viruses to increase the amount of virus produced by the cell.

24. The method of claim 23, wherein the one or more viruses are comprised in a non-replicating viral vector.

25. The method of claim 24, wherein the viral vector is an adenovirus (Ad), an adeno-associated virus (AAV) or lentivirus (LV).

26. The method of claim 24, wherein the viral vector is administered to the cell for transduction of the cell and the one or more vanadium compounds increase permissiveness of the cell to the viral vector to increase the amount of viral transduction.

27. The method of claim 24, wherein the viral vector comprises a therapeutic gene and one or more vanadium compounds increase permissiveness of the cell to the viral vector to increase the amount of virally-encoded therapeutic gene incorporated into the genome of the cell.

28. The method of claim 20, wherein the one or more viruses are administered to the cell to treat a disease, disorder or condition in the cell and the one or more vanadium compounds increase permissiveness of the cell to the one ore more viruses to increase the efficacy of the for treating the disease, disorder or condition.

29. (canceled)

30. (canceled)
